(19) United States
(12) Patent Application Publication
(10) Pub. No.: US 2013/0253321 A1
(43) Pub. Date: Sep. 26, 2013

(54) ULTRASONIC DIAGNOSTIC APPARATUS, IMAGE PROCESSING APPARATUS, AND IMAGE PROCESSING METHOD

(71) Applicants: KABUSHIKI KAISHA TOSHIBA, Tokyo (JP); TOSHIBA MEDICAL SYSTEMS CORPORATION, Otawara-shi (JP)

(72) Inventors: Shinichi HASHIMOTO, Otawara-shi (JP); Kenji Hamada, Otawara-shi (JP)

(73) Assignees: Toshiba Medical Systems Corporation, Otawara-shi (JP); Kabushiki Kaisha Toshiba, Tokyo (JP)

(21) Appl. No.: 13/848,246

(22) Filed: Mar. 21, 2013

(30) Foreign Application Priority Data
Mar. 21, 2012 (JP) 2012-063560

(51) Int. Cl. A61B 8/14

(52) U.S. Cl. CPC A61B 8/145
USPC 600/443

(57) ABSTRACT

An ultrasonic diagnostic apparatus according to an embodiment includes an ultrasonic probe, a storage controller, and an output controller. The ultrasonic probe is configured to perform three-dimensional ultrasonic scanning under transmission/reception control. The storage controller is configured to control data generated by the three-dimensional scanning performed by the ultrasonic probe so as to be stored in a predetermined memory as a plurality of pieces of two-dimensional data generated by two-dimensionally scanning a plurality of predetermined cross-sections each of whose positions is continuously changed along a predetermined direction in a region of the three-dimensional scanning. The output controller is configured to control a plurality of pieces of two-dimensional image data based on the pieces of two-dimensional data stored in the predetermined memory so as to be output as moving image data to a predetermined output unit.

### CROSS-REFERENCE TO RELATED APPLICATIONS

This application is based upon and claims the benefit of priority from Japanese Patent Application No. 2012-063560 filed on Mar. 21, 2012, the entire contents of all of which are incorporated herein by reference.

### FIELD

Embodiments described herein relate generally to an ultrasonic diagnostic apparatus, an image processing apparatus, and an image processing method.

### BACKGROUND

In recent years, ultrasonic diagnostic apparatuses are in practical use that uses an ultrasonic probe that can perform three-dimensional ultrasonic scanning to generate three-dimensional ultrasonic image data (volume data) and to display a two-dimensional image based on the volume data. Among the ultrasonic probes that can perform three-dimensional ultrasonic scanning, there are a mechanical 4D probe that performs three-dimensional scanning by mechanically oscillating a plurality of vibrators arranged in a line for two-dimensional scanning and a 2D array probe that electronically performs three-dimensional scanning by using a plurality of vibrators arranged in a grid-like pattern.

Such an ultrasonic diagnostic apparatus reconstructs from the volume data, for example, a multiplaner reconstruction (MPR) image of a predetermined section in a three-dimensionally scanned region as an image for displaying the volume data. However, the image for display generated from the volume data has been subjected to degradation in image quality in some cases.

### BRIEF DESCRIPTION OF THE DRAWINGS

FIG. 1 is a diagram for explaining a configuration example of a conventional ultrasonic diagnostic apparatus;

FIG. 2 is a diagram for explaining a conventional data management unit;

FIG. 3 is a diagram for explaining a configuration example of an ultrasonic diagnostic apparatus according to a first embodiment;

FIG. 4 is a diagram for explaining processing by a controller according to the first embodiment;

FIG. 5 is a flowchart for explaining processing by the ultrasonic diagnostic apparatus according to the first embodiment;

FIGS. 6, 7, and 8 are diagrams for explaining a second embodiment;

FIG. 9 is a flowchart for explaining processing by an ultrasonic diagnostic apparatus according to the second embodiment;

FIG. 10 is a diagram for explaining a third embodiment;

FIG. 11 is a flowchart for explaining processing by an ultrasonic diagnostic apparatus according to the third embodiment;

FIG. 12 is a diagram for explaining a fourth embodiment;

FIG. 13 is a flowchart for explaining processing by an ultrasonic diagnostic apparatus according to the fourth embodiment;

FIG. 14 is a diagram for explaining a fifth embodiment; and

FIG. 15 is a flowchart for explaining processing by an ultrasonic diagnostic apparatus according to the fifth embodiment.

### DETAILED DESCRIPTION

An ultrasonic diagnostic apparatus according to an embodiment includes an ultrasonic probe, a storage controller, and an output controller. The ultrasonic probe is configured to perform three-dimensional ultrasonic scanning under transmission/reception control. The storage controller is configured to control data generated by the three-dimensional scanning performed by the ultrasonic probe so as to be stored in a predetermined memory as a plurality of pieces of two-dimensional data generated by two-dimensionally scanning a plurality of predetermined cross-sections each of whose positions is continuously changed along a predetermined direction in a region of the three-dimensional scanning. The output controller is configured to control a plurality of pieces of two-dimensional image data based on the pieces of two-dimensional data stored in the predetermined memory so as to be output as moving image data to a predetermined output unit.

Embodiments of an ultrasonic diagnostic apparatus will be described below in detail with reference to the accompanying drawings.

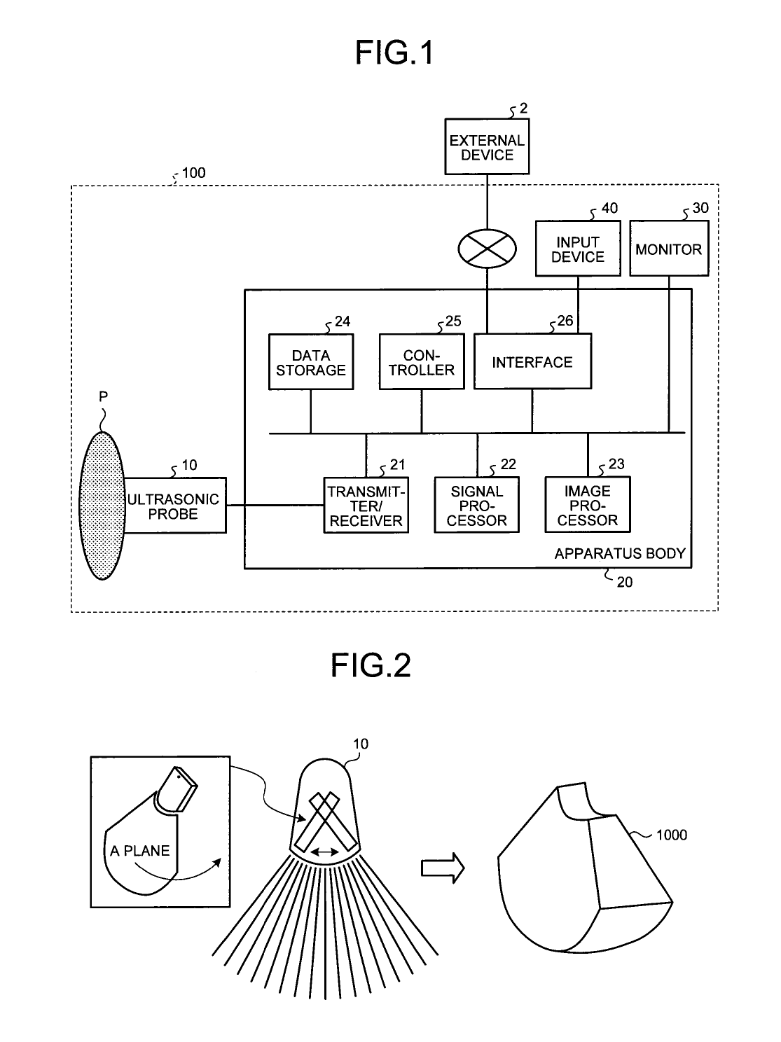

First, before the ultrasonic diagnostic apparatus according to a first embodiment is described, a description will be made of a conventional ultrasonic diagnostic apparatus using FIG. 1. FIG. 1 is a diagram for explaining a configuration example of the conventional ultrasonic diagnostic apparatus. As illustrated in FIG. 1, this conventional ultrasonic diagnostic apparatus 100 includes an ultrasonic probe 10, an apparatus body 20, a monitor 30, and an input device 40. The apparatus body 20 included in the ultrasonic diagnostic apparatus 100 is connected to an external device 2 via a network, as illustrated in FIG. 1.

The ultrasonic probe 10 includes, for example, a plurality of piezoelectric vibrators as a plurality of acoustic elements (an acoustic element group). The piezoelectric vibrators generate ultrasonic waves based on driving signals supplied from a transmitter/receiver 21 included in the apparatus body 20 (to be described later). The ultrasonic probe 10 receives reflected waves from a subject and converts them into electric signals. The ultrasonic probe 10 also includes a matching layer provided at the piezoelectric vibrators, a backing material that prevents the ultrasonic waves from propagating backward from the piezoelectric vibrators, and the like.

When ultrasonic waves are transmitted from the ultrasonic probe 10 to a subject P, the transmitted ultrasonic waves are continuously reflected on discontinuous planes of acoustic impedance in the body tissue of the subject P, and are received as a reflected-wave signal at the piezoelectric vibrators included in the ultrasonic probe 10. The amplitude of the received reflected-wave signal depends on a difference in acoustic impedance at the discontinuous surface on which the ultrasonic waves are reflected. Note that, the reflected-wave signal resulting from reflection of the transmitted ultrasonic pulses on a moving blood flow, the surface of a heart wall, or the like is subjected to a frequency shift due to the Doppler effect, depending on the velocity component of the moving object in the direction of transmission of the ultrasonic waves.

The ultrasonic probe 10 illustrated in FIG. 1 is an ultrasonic probe that can scan the subject P both two-dimensionally and three-dimensionally using ultrasonic waves. The ultrasonic probe 10 illustrated in FIG. 1 performs three-dimensional ultrasonic scanning under transmission/reception control. In other words, the ultrasonic probe 10 illustrated in FIG. 1 performs the three-dimensional ultrasonic scanning not by being manually moved by an operator while performing two-dimensional scanning, but with mechanical automatic control by the transmitter/receiver 21 and the like (to be described later). Specifically, the ultrasonic probe 10 illustrated in FIG. 1 performs the three-dimensional scanning by oscillating the vibrator group arranged in a line. More specifically, the ultrasonic probe 10 illustrated in FIG. 1 is a mechanical 4D probe that two-dimensionally scans the subject P by using the piezoelectric vibrators (vibrator group) arranged in a line and mechanically oscillates the piezoelectric vibrators between predetermined angles (oscillation angles) so as to perform the three-dimensional ultrasonic scanning.

The input device 40 includes a mouse, a keyboard, buttons, panel switches, a touch command screen, a foot switch, a trackball, a joystick, and the like. The input device 40 accepts various setting requests from the operator of the ultrasonic diagnostic apparatus 100, and transfers the thus accepted various setting requests to the apparatus body 20.

The monitor 30 displays a graphical user interface (GUI) for the operator of the ultrasonic diagnostic apparatus 100 to enter the various setting requests using the input device 40, and displays, for example, an ultrasonic image generated in the apparatus body 20.

The apparatus body 20 is an apparatus that performs overall control of ultrasonic imaging, and specifically, an apparatus that generates ultrasonic image data based on the reflected waves received by the ultrasonic probe 10. The apparatus body 20 includes, for example, as illustrated in FIG. 1, the transmitter/receiver 21, a signal processor 22, an image processor 23, a data storage 24, a controller 25, and an interface 26.

The transmitter/receiver 21 controls the ultrasonic probe 10 to perform the three-dimensional ultrasonic scanning. The transmitter/receiver 21 includes a trigger generating circuit, a transmission delay circuit, a pulser circuit, and the like, and supplies the driving signals to the ultrasonic probe 10. The pulser circuit repeatedly generates rate pulses for forming a transmission ultrasonic wave at a predetermined rate frequency. The transmission delay circuit gives each of the rate pulses generated by the pulser circuit a delay time for each of the piezoelectric vibrators necessary for focusing the ultrasonic waves generated from the ultrasonic probe 10 into beam-like waves and determining transmission directivity. The trigger generating circuit applies the driving signals (driving pulses) to the ultrasonic probe 10 at timing based on the rate pulses. In other words, the delay circuit changes the delay time given to each of the rate pulses to arbitrarily adjust the directions of transmission from the piezoelectric vibrator surfaces.

Note that the transmitter/receiver 21 has a function that can instantly change a transmission frequency, a transmission driving voltage, and the like in order to perform a predetermined scan sequence based on an instruction of the controller 25 (to be described later). The change in the transmission driving voltage is specifically achieved by a linear amplifier type transmission circuit that can instantly change the value thereof, or by a mechanism that electrically switches between a plurality of power supply units.

The transmitter/receiver 21 includes an amplifier circuit, an analog/digital (A/D) converter, an adder, a phase detection circuit, and the like, and applies various types of processing to the reflected-wave signal received by the ultrasonic probe 10 to generate reflected-wave data. The amplifier circuit performs gain correction processing of amplifying the reflected-wave signal for each channel. The A/D converter applies A/D conversion to the gain-corrected reflected-wave signal, and gives the converted digital data a delay time necessary for determining reception directivity. The adder applies addition processing to the reflected-wave signal which has been processed by the A/D converter. The addition processing by the adder enhances a reflection component in a direction corresponding to the reception directivity of the reflected-wave signal. The phase detection circuit converts an output signal of the adder into an in-phase signal (I signal) and a quadrature-phase signal (Q signal) in a baseband. The phase detection circuit then outputs the I signal and the Q signal (IQ signal) to the signal processor 22 at a subsequent stage. Note that the data before being processed by the phase detection circuit is also called an RF signal. Hereinafter, the IQ signal and RF signal which are generated based on the reflected-wave of the ultrasonic wave are collectively mentioned as the reflected-wave data.

In this manner, the transmitter/receiver 21 controls the transmission directivity and the reception directivity in the transmission/reception of the ultrasonic waves. In other words, the transmitter/receiver 21 serves as a transmit beamformer and as a receive beamformer. Here, the transmitter/receiver 21 transmits a two-dimensional ultrasonic beam from the vibrator group of the ultrasonic probe 10 serving as a mechanical 4D probe so as to perform the two-dimensional scanning (scanning of a cross-section) of the subject P. Based on this scanning, the transmitter/receiver 21 generates two-dimensional reflected-wave data.

In addition, the transmitter/receiver 21 oscillates the vibrator group of the ultrasonic probe 10 serving as a mechanical 4D probe at a predetermined oscillation speed in a predetermined range so as to perform the three-dimensional scanning by performing the two-dimensional scanning of a plurality of cross-sections. When the three-dimensional scanning is performed, the transmitter/receiver 21 generates three-dimensional reflected-wave data from reflected-wave signals of the respective cross-sections. Note that the operator set the oscillation angles (oscillation range) so as to set a range in which the three-dimensional scanning is performed.

The signal processor 22 receives the reflected-wave data from the transmitter/receiver 21, and through applying logarithmic amplification, envelope detection processing, and the like to the received reflected-wave data, generates data (B-mode data) in which a signal strength is expressed by the brightness of luminance. The signal processor 22 also applies frequency analysis to velocity information in the reflected-wave data received from the transmitter/receiver 21, and through extracting a blood flow component, a tissue component, and a contrast medium echo component that are due to the Doppler effect, generates data (Doppler data) of moving object information, such as an average velocity, a variance, and a power, extracted at multiple points.

The signal processor 22 can process both the two-dimensional reflected-wave data and the three-dimensional reflected-wave data. Specifically, the signal processor 22 generates two-dimensional B-mode data from the two-dimensional reflected-wave data and three-dimensional B-mode data from the three-dimensional reflected-wave data. The signal processor 22 also generates two-dimensional Doppler data from the two-dimensional reflected-wave data and three-dimensional Doppler data from the three-dimensional reflected-wave data.

The image processor 23 generates the ultrasonic image data from the data generated by the signal processor 22. Specifically, the image processor 23 generates, from the B-mode data, B-mode image data in which the strength of the reflected-wave is expressed by luminance. Also, the image processor 23 generates, from the Doppler data, Doppler image data, such as an average velocity image, a variance image, a power image, and a combined image thereof, representing the moving object information. The image processor 23 can also generate a composite image obtained by combining the ultrasonic image with text information of various parameters, scales, body marks (pictograms), and the like.

The image processor 23 scan-converts a scanning line signal row of the ultrasonic scan into a scanning line signal row in a video format represented by that for a television or the like, and generates ultrasonic image data as an image for display. The image processor 23 also performs various types of image processing other than the scan conversion, such as image processing (smoothing processing) of regenerating an image having an average luminance by using a plurality of image frames scan-converted and image processing (edge reinforcement processing) that uses a differential filter in an image.

In other words, the B-mode data and the Doppler data are ultrasonic image data before the scan conversion processing whereas the data generated by the image processor 23 is ultrasonic image data for display after the scan conversion processing. Note that the B-mode data and the Doppler data are also called raw data.

Moreover, the image processor 23 applies coordinate transformation to the three-dimensional B-mode data generated by the signal processor 22 so as to generate three-dimensional B-mode image data. The image processor 23 also applies coordinate transformation to the three-dimensional Doppler data generated by the signal processor 22 so as to generate three-dimensional color Doppler image data. In other words, the image processor 23 generates the three-dimensional B-mode image data and the three-dimensional color Doppler image data, as volume data which is three-dimensional ultrasonic image data.

Furthermore, in order to generate various types of two-dimensional image data for displaying the volume data on the monitor 30, the image processor 23 applies rendering processing to the volume data. Examples of the rendering processing performed by the image processor 23 include processing using a multiplaner reconstruction (MPR) method to generate MPR image data from the volume data, processing of performing curved MPR on the volume data, processing of performing intensity projection on the volume data, and volume rendering (VR) processing of generating two-dimensional image data reflecting three-dimensional information.

The data storage 24 stores therein various types of data generated in the apparatus body 20. The data storage 24 stores therein, for example, the reflected-wave data generated by the transmitter/receiver 21, the B-mode data and the Doppler data generated by the signal processor 22, and the ultrasonic image data generated by the image processor 23. The data storage 24 also stores therein the three-dimensional reflected-wave data, the three-dimensional B-mode data, the three-dimensional Doppler data, and the three-dimensional ultrasonic image data.

The controller 25 is a control processor (central processing unit (CPU)) that implements a function as an information processing device, and controls overall processing of the ultrasonic diagnostic apparatus 100. Specifically, based on the various setting requests entered by the operator via the input device 40, various control programs, and various types of data, the controller 25 controls the processing of the transmitter/receiver 21, the signal processor 22, and the image processor 23. The controller 25 also controls the data storage processing into the data storage 24. The controller 25 further performs output control of the data stored in the data storage 24. For example, the controller 25 performs the control so as to display the ultrasonic image data or the like on the monitor 30.

The interface 26 is an interface to the input device 40 and the external device 2. For example, the interface 26 transfers, to the controller 25, various types of setting information and various instructions accepted by the input device 40 from the operator. The interface 26 can also output, for example, the image data generated in the apparatus body 20 to the external device 2 via the network.

The external device 2 is a device connected to the apparatus body 20 via the interface 26. The external device 2 is, for example, a database for a picture archiving and communication system (PACS) which is a system for managing various data of medical images, or a database for an electronic chart system for managing electronic charts with medical images attached. Alternatively, the external device 2 is, for example, a workstation or a personal computer (PC) used for image interpretation by a medical doctor or an inspection engineer working in a hospital. Alternatively, the external device 2 is a printer, or a non-transitory storage medium such as a CD or a DVD. The controller 25 controls output processing of the various types of data stored in the data storage 24 to the external device 2.

As described above, the ultrasonic probe 10 is a mechanical 4D probe that performs the three-dimensional ultrasonic scanning by mechanically oscillating the vibrator group serving as a 2D scanning probe, and the conventional ultrasonic diagnostic apparatus 100 is a three-dimensional ultrasonic diagnostic apparatus that collects the volume data by using the ultrasonic probe 10. The mechanical 4D probe mechanically oscillates the vibrator group only when collecting the volume data. The conventional ultrasonic diagnostic apparatus 100 starts the mechanical oscillation, and also starts generating and collecting three-dimensional data. Here, the three-dimensional data refers to the three-dimensional reflected-wave data, the three-dimensional signal-processed data (three-dimensional B-mode data and three-dimensional Doppler data), the volume data (three-dimensional B-mode image data and three-dimensional Doppler image data), and the like.

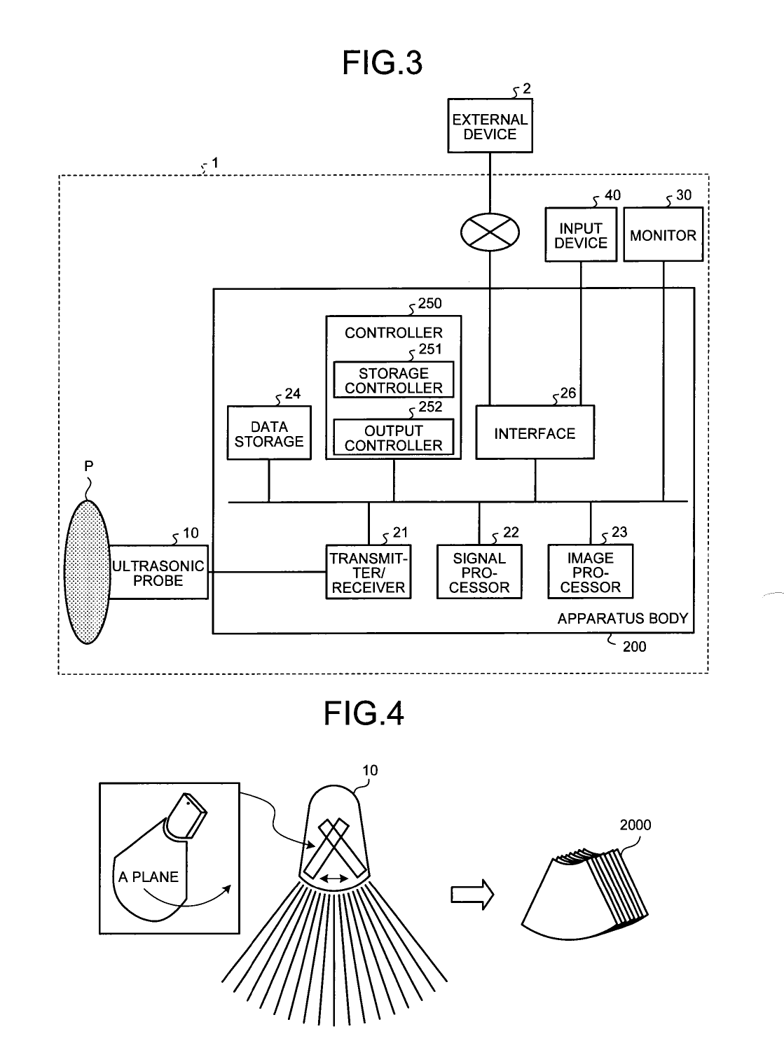

As described above, the reflected-wave data generated in the transmitter/receiver 21 is processed into the volume data through the signal processing by the signal processor 22 and the image processing by the image processor 23. In the conventional ultrasonic diagnostic apparatus 100, data in the case of the three-dimensional scanning is typically managed in management units of one-scan data in a three-dimensional scan range. FIG. 2 is a diagram for explaining the conventional data management unit.

Specifically, as illustrated in FIG. 2, the conventional ultrasonic diagnostic apparatus 100 manages, as a unit of handling in cases of data storage and data read, the three-dimensional data resulting from one swing of oscillation of the vibrator group included in the ultrasonic probe 10 in the three-dimensional scan range. For example, conventionally, as illustrated in FIG. 2, the ultrasonic diagnostic apparatus 100 manages one piece of volume data 1000 generated by the image processor 23 through one swing of oscillation, as one piece of data. Note that, although not illustrated, the conventional ultrasonic diagnostic apparatus 100 also manages, as one piece of data, the three-dimensional reflected-wave data generated by the transmitter/receiver 21 through one swing of oscillation and the three-dimensional signal-processed data generated by the signal processor 22 through one swing of oscillation.

Also, conventionally, in order to observe the volume data which is three-dimensional ultrasonic image data, the image processor 23 generates a VR image and an MPR image from the volume data. In the case of observing the MPR image in an ultrasonic examination, observation is mainly made at three orthogonal cross-sections, each called an A plane, a B plane, and a C plane. Description will be made below of the A, B, and C planes used in the ultrasonic probe 10 serving as a mechanical 4D probe.

The A plane refers to a cross-section defined by the direction of arrangement of the vibrator group in the ultrasonic probe 10 and by the direction of transmission of the ultrasonic beam (refer to FIG. 2). In other words, the A plane is a cross-section approximate to a cross-section two-dimensionally scanned by the ultrasonic probe 10. The B plane refers to a cross-section defined by the direction of transmission of the ultrasonic beam and by the direction of oscillation. In other words, the direction of oscillation is in the direction of the B plane. The C plane refers to a cross-section orthogonal to the A and B planes, that is, a cross-section in the orthogonal direction to the direction of transmission of the ultrasonic beam.

The three-dimensional scanning using the ultrasonic probe 10 serving as a mechanical 4D probe is performed by mechanically oscillating a vibrator group suitable for collecting two-dimensional ultrasonic image data obtained by two-dimensionally scanning cross-sections corresponding to the A plane. When the three-dimensional scanning is performed, a higher mechanical oscillation speed increases the rate of iterative collection of three-dimensional data, which is called a volume rate, and thus makes it possible to update an image based on the volume data at a high speed. Thus, in order to improve real-time performance, the mechanical oscillation speed needs to be increased. When the mechanical oscillation speed is increased, however, the scanning line density in the direction of oscillation needs to be reduced in order to ensure the volume rate. Thus, in the case in which an image based on the volume data is updated at several frames per second, the image quality of MPR images in the B and C planes is typically lower than the image quality of an MPR image in the A plane. For this reason, the A plane has been conventionally often used for observation of the MPR image.

The image quality of the MPR images in the B and C planes markedly drops particularly when the heart of a fetus is observed using the ultrasonic probe 10 serving as a mechanical 4D probe. A fetus has a heart rate of, for example, 120 beats per minute, which is higher than that of an adult. When the heart of the fetus is three-dimensionally scanned at a normal oscillation speed, therefore, pieces of data collected from different positions have different cardiac phases. Thus, one piece of volume data collected by once performing the three-dimensional scanning contains the fetal heart at various cardiac phases.

Thus, there is known a technique of collecting, as three-dimensional moving image data, the volume data at each cardiac phase of the fetal heart from the data collected by three-dimensionally scanning the entire fetal heart with the ultrasonic probe 10 at a low speed (hereinafter called a fetal heart observation technique). The fetal heart observation technique collects a plurality of two-dimensional cross-sectional images by once three-dimensionally scanning the fetal heart having a high heart rate at a low speed, and arranges the collected two-dimensional cross-sectional images in chronological order in the direction of oscillation. By performing the low-speed oscillation, it is possible to continuously collect the two-dimensional cross-sectional images at cardiac phases within one period while the vibrator group swings by a small angle (such as three degrees). The cardiac phase of each of the two-dimensional cross-sectional images can be obtained by applying frequency analysis to reflected-wave data that has generated each of the two-dimensional cross-sectional images. Based on the result of the frequency analysis, the fetal heart observation technique arranges the two-dimensional cross-sectional images at the same cardiac phase along the direction of oscillation so as to reconstruct the volume data at the same cardiac phase. With this, the fetal heart observation technique can collect the three-dimensional moving image data along the cardiac phases of the fetal heart by once performing the three-dimensional scanning.

In the fetal heart observation technique, however, influences of accuracy of heart rate detection with the frequency analysis, fetal movement, and the like are reflected in the volume data after being reconstructed. This is likely to further degrade the image quality of the MPR images in the B and C planes than in the case of the three-dimensional scanning at a normal oscillation speed. In this manner, in the three-dimensional scanning by the conventional ultrasonic diagnostic apparatus 100, the image quality of the MPR images in the B and C planes degrades.

Furthermore, in the conventional ultrasonic diagnostic apparatus 100, when an MPR image is reconstructed from the volume data by setting, as the A plane, a cross-section different from the cross-section scanned by the ultrasonic beam, the image quality of the MPR image in the A plane also degrades. This is because the volume data is conventionally stored as a management unit in the case of performing the three-dimensional scanning. Thus, the image quality of the MPR image in the A plane further degrades particularly as the scanned cross-section is farther separated from the A plane for reconstruction.

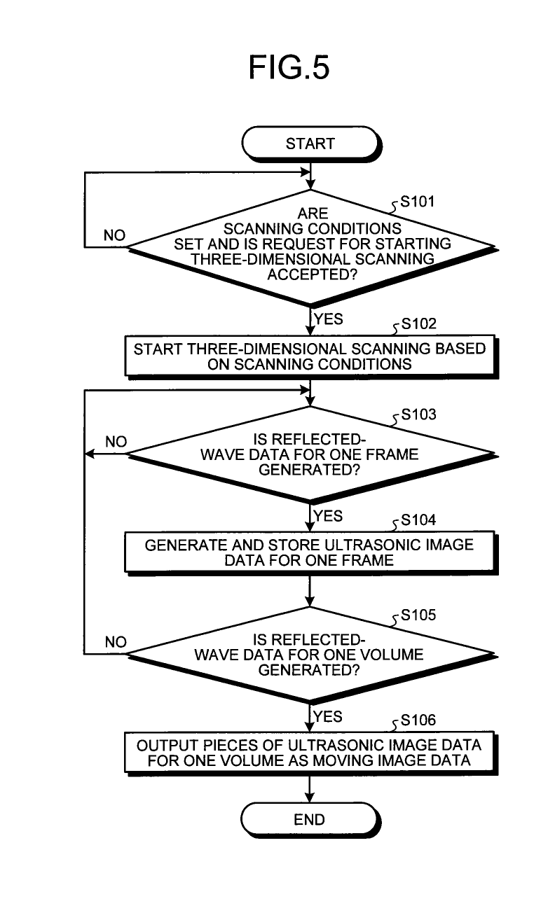

In order to avoid the degradation in the image quality of the image displayed by the three-dimensional ultrasonic scanning, the ultrasonic diagnostic apparatus according to the first embodiment performs processing described below. FIG. 3 is a diagram for explaining a configuration example of the ultrasonic diagnostic apparatus according to the first embodiment.

As illustrated in FIG. 3, in the same manner as the above-described conventional ultrasonic diagnostic apparatus 100, this ultrasonic diagnostic apparatus 1 according to the first embodiment includes the ultrasonic probe 10 serving as a mechanical 4D probe, the monitor 30, and the input device 40. Specifically, the ultrasonic probe 10 performs the three-dimensional ultrasonic scanning under the transmission/reception control. Specifically, the ultrasonic probe 10 performs the three-dimensional scanning by mechanically oscillating the vibrator group arranged in a line while the vibrator group performs the two-dimensional scanning. The ultrasonic diagnostic apparatus 1 according to the first embodiment includes an apparatus body 200 instead of the apparatus body 20 included in the conventional ultrasonic diagnostic apparatus 100. The apparatus body 200 is connected to the above-described external device 2 via the network or the like, as illustrated in FIG. 3.

In the same manner as the apparatus body 20 described using FIG. 1, the apparatus body 200 illustrated in FIG. 3 includes the transmitter/receiver 21, the signal processor 22, the image processor 23, the data storage 24, and the interface 26. The transmitter/receiver 21, the signal processor 22, the image processor 23, the data storage 24, and the interface 26 included in the apparatus body 200 illustrated in FIG. 3 performs the same processing as that of the respective units in the apparatus body 20 described using FIG. 1. As compared with the apparatus body 20 described using FIG. 1, the apparatus body 200 illustrated in FIG. 3 includes a controller 250 instead of the controller 25.

In the same manner as the controller 25, the controller 250 is a CPU that implements a function as an information processing device, and controls overall processing of the ultrasonic diagnostic apparatus 1. The controller 250 performs the same control processing as that of the controller 25 except storage control and output control according to the first embodiment which are to be described below. That is to say, the ultrasonic diagnostic apparatus 1 is a three-dimensional ultrasonic diagnostic apparatus configured in a manner similar to the ultrasonic diagnostic apparatus 100. Note, however, that the controller 250 includes, as illustrated in FIG. 3, a storage controller 251 that performs the storage control according to the first embodiment and an output controller 252 that performs the output control according to the first embodiment.

The storage controller 251 controls the data generated by the three-dimensional scanning performed by the ultrasonic probe 10 to be stored in the data storage 24 as a plurality of pieces of two-dimensional data generated by two-dimensionally scanning a plurality of predetermined cross-sections each of whose positions is continuously changed along a predetermined direction in a region of the three-dimensional scanning. The data generated by the three-dimensional scanning is three-dimensional data, such as the three-dimensional reflected-wave data, the three-dimensional signal-processed data (three-dimensional B-mode data and three-dimensional Doppler data), and the volume data (three-dimensional B-mode image data and three-dimensional Doppler image data). The output controller 252 controls a plurality of pieces of two-dimensional image data based on the pieces of two-dimensional data stored in the data storage 24 to be output as moving image data to a predetermined output unit (the monitor 30 or the external device 2).

Specifically, in the first embodiment, the storage controller 251 controls the pieces of two-dimensional image data so as to be stored in the data storage 24 as a plurality of pieces of two-dimensional data. In the first embodiment, the output controller 252 controls the pieces of two-dimensional image data stored in the data storage 24 so as to be output as moving image data to the predetermined output unit.

Here, the term “predetermined cross-section” refers to the A plane defined by the direction of arrangement of the vibrator group in the ultrasonic probe 10 and by the direction of transmission of the ultrasonic beam. The expression “a plurality of predetermined cross-sections each of whose positions is continuously changed along a predetermined direction” refers to a plurality of A planes each of whose positions where the two-dimensional scanning is applied is continuously changed along the direction of the oscillation.

The term “two-dimensional data” refers to the two-dimensional reflected-wave data, two-dimensional signal-processed data, and two-dimensional ultrasonic image data of the A plane two-dimensionally scanned. The term “two-dimensional image data” refers to two-dimensional B-mode image data or two-dimensional Doppler image data which is the two-dimensional ultrasonic image data.

That is to say, in the first embodiment, when the three-dimensional scanning is performed by continuously changing the position of two-dimensional scanning along the direction of oscillation, the data is managed in management units of two-dimensional data instead of conventional three-dimensional data. Specifically, assuming the three-dimensionally scanned region as the two-dimensionally scanned A planes, the storage controller 251 manages one piece of three-dimensional data as a two-dimensional data group composed of a plurality of pieces of two-dimensional data each corresponding to one of the A planes. The storage controller 251 controls the transmitter/receiver 21 so as to generate a two-dimensional reflected-wave data group as a plurality of pieces of two-dimensional data each corresponding to one of the A planes. The storage controller 251 also controls the signal processor 22 so as to generate a two-dimensional signal-processed data group as a plurality of pieces of two-dimensional data each corresponding to one of the A planes. The storage controller 251 further controls the image processor 23 so as to generate a two-dimensional image data group as a plurality of pieces of two-dimensional data each corresponding to one of the A planes.

Then, in the first embodiment, the storage controller 251 controls each of the pieces of two-dimensional image data of the A planes generated by the image processor 23 so as to be stored in the data storage 24. The output controller 252 displays on the monitor 30, or outputs to the external device 2, the pieces of two-dimensional image data stored in the data storage 24, as moving image data. In other words, the controller 250 according to the first embodiment controls the data collected by the three-dimensional scanning so as to be treated as moving image data of the two-dimensional image data.

An example of the above-described control processing will be described below. First, the operator of the ultrasonic diagnostic apparatus 1 sets in advance, via the input device 40, scanning conditions for performing the three-dimensional scanning. Specifically, in order to set a range in which the three-dimensional scanning is to be performed, the operator sets in advance oscillation angles (an angle corresponding to a position at one end of an oscillation range and an angle corresponding to a position at the other end of the oscillation range). The operator of the ultrasonic diagnostic apparatus 1 also sets in advance an oscillation speed, or an oscillation time required for one swing of oscillation.

The operator sets the scanning conditions so as to change the position of each of the predetermined cross-sections (A planes) by increments of constant amount. Specifically, the operator sets the scanning conditions so as to change the position of the A plane by increments of constant angle (constant distance) in a constant time duration. In other words, the operator sets the scanning conditions so that the oscillation speed is constant.

Then, the operator determines the position of the ultrasonic probe 10 so that an organ to be observed can be three-dimensionally scanned while included in a region composed of desired A planes. Then, the operator issues a start request to start the control processing of the controller 250 according to the first embodiment by, for example, pressing a switch (2D moving image data storing switch) included in the input device 40. The vibrator group embedded in the ultrasonic probe 10 is normally fixed in the center position when the three-dimensional scanning is not being performed. Pressing the 2D moving image data storing switch moves the position of the vibrator group to one end of the oscillation range under control of the controller 250.

Then, the vibrator group starts to mechanically oscillate, and the two-dimensional image data starts to be collected. The image processor 23 generates the two-dimensional image data according to an acoustic frame rate determined by the scanning conditions set for the vibrator group. Under control of the storage controller 251, the image processor 23 stores the two-dimensional image data generated at the acoustic frame rate in a space of a conventional cine memory allocated in the data storage 24. Note that the storage controller 251 may perform the control so as to store the two-dimensional image data in the space of the cine memory at an image capture rate that is feasible with the performance of hardware included in the ultrasonic diagnostic apparatus 1, for example, at a frame rate at which the monitor 30 can perform display.

Then, at the time when the mechanically oscillated vibrator group reaches the other end of the oscillation range, the controller 250 stops the oscillation. Note that, at the time when one swing of oscillation finishes, the operator presses a freeze button included in the input device 40 to stop the data collection. Then, after the operator presses again the 2D moving image data storing switch, the storage controller 251 notifies the output controller 252 that the pieces of two-dimensional image data stored in the data storage 24 can be output as two-dimensional moving image data obtained by the three-dimensional scanning. Note that the first embodiment may be a case in which, at the time when one swing of oscillation finishes, the operator does not press the freeze button or the 2D moving image data storing switch, but the storage controller 251 automatically starts the processing thereof.

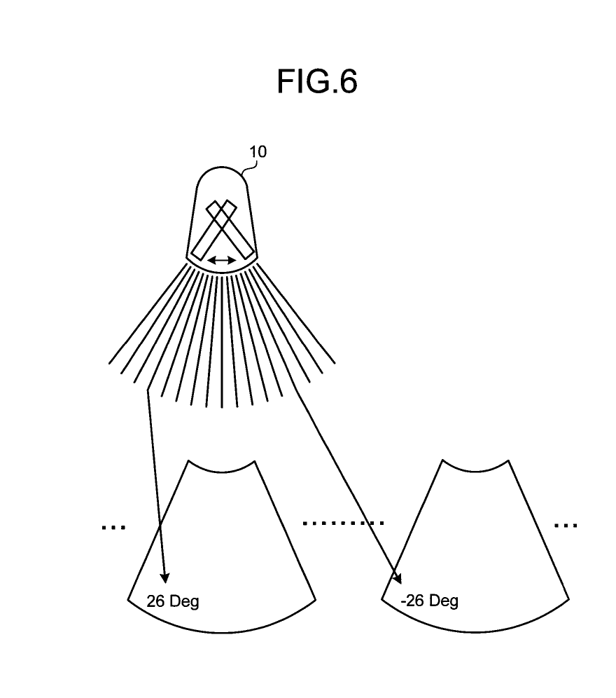

FIG. 4 is a diagram for explaining the processing by the controller according to the first embodiment. As illustrated in FIG. 4, the ultrasonic probe 10 swings, along the direction of oscillation (direction of the B plane), the vibrator group which two-dimensionally scans the cross-sections corresponding to the A planes, and thus once performs the three-dimensional scanning. Under control of the storage controller 251, the image processor 23 generates pieces of two-dimensional ultrasonic image data of the A planes (a two-dimensional image data group 2000 illustrated in FIG. 4) as three-dimensional ultrasonic image data in the three-dimensionally scanned range. Then, under control of the storage controller 251, the image processor 23 stores the two-dimensional image data group 2000 in the data storage 24 (cine memory).

In this manner, the storage controller 251 treats, as one unit of performing the storage control, a range in which the position of the two-dimensional scanning on each of the predetermined cross-sections (A planes) is changed. In other words, the storage controller 251 manages the pieces of two-dimensional image data collected through one swing of oscillation collectively as one unit. Note that, in order to easily manage the pieces of two-dimensional image data collected through one swing of oscillation as one unit, the storage controller 251 refreshes and clears the cine memory in the data storage 24 before the position of the vibrator group moves to the start position of the oscillation scanning by the 2D moving image data storing switch is pressed. Then, the storage controller 251 starts the storage control of the two-dimensional image data. With this, the output controller 252 can recognize that the two-dimensional image data first stored in the data storage 24 is image data corresponding to the oscillation start position, and that the two-dimensional image data last stored in the data storage 24 is image data corresponding to the oscillation end position.

Then, the output controller 252, for example, reads the two-dimensional image data group 2000 from the data storage 24 and controls the monitor 30 to dynamically display the two-dimensional image data group 2000 serving as moving image data as a movie. Alternatively, the output controller 252, for example, reads the two-dimensional image data group 2000 from the data storage 24 and controls the monitor 30 to display frames constituting the two-dimensional image data group 2000 serving as moving image data, as thumbnails.

Still alternatively, the output controller 252, for example, reads the two-dimensional image data group 2000 from the data storage 24 and outputs the two-dimensional image data group 2000 as moving image data to the external device 2.

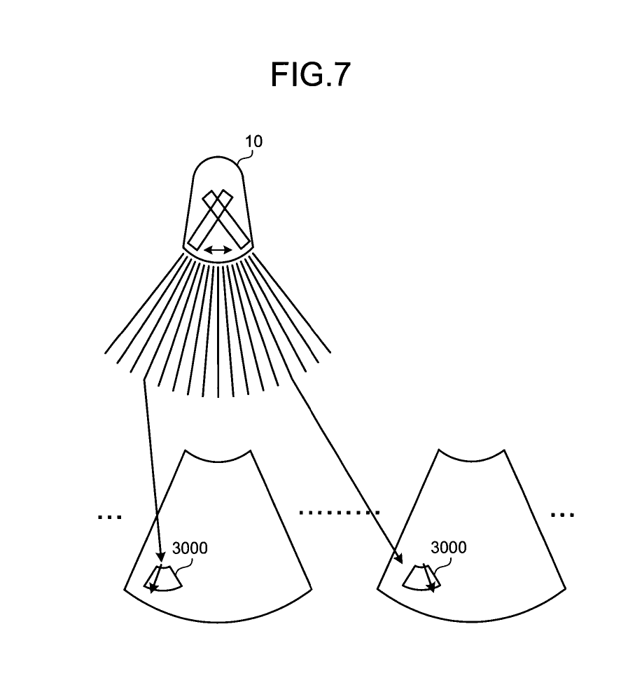

Next, processing by the ultrasonic diagnostic apparatus according to the first embodiment will be described using FIG. 5. FIG. 5 is a flowchart for explaining the processing by the ultrasonic diagnostic apparatus according to the first embodiment.

As illustrated in FIG. 5, the ultrasonic diagnostic apparatus 1 according to the first embodiment determines whether the scanning conditions are set and a request for starting the three-dimensional scanning is accepted (Step S101). If the request for starting the three-dimensional scanning is not accepted (No at Step S101), the ultrasonic diagnostic apparatus 1 waits until the request for starting the three-dimensional scanning is accepted.

If, instead, the request for starting the three-dimensional scanning is accepted (Yes at Step S101), the ultrasonic probe 10 starts, under control of the controller 250, the three-dimensional scanning based on the scanning conditions (Step S102). Then, the storage controller 251 determines whether reflected-wave data for one frame is generated (Step S103). If the reflected-wave data for one frame is not generated (No at Step S103), the storage controller 251 waits until the reflected-wave data for one frame is generated.

If, instead, the reflected-wave data for one frame is generated (Yes at Step S103), the image processor 23 generates, under control of the storage controller 251, ultrasonic image data for one frame and stores the generated data in the data storage 24 (Step S104). Then, the storage controller 251 determines whether the reflected-wave data for one volume is generated (Step S105). If the reflected-wave data for one volume is not generated (No at Step S105), the storage controller 251 returns to Step S103 and determines whether the reflected-wave data for one frame is generated.

If, instead, the reflected-wave data for one volume is generated (Yes at Step S105), the output controller 252 outputs a plurality of pieces of ultrasonic image data for one volume (two-dimensional ultrasonic image data group) as moving image data (Step S106), and terminates the processing. Note that the first embodiment may be a case in which the processing from Step S103 to Step S106 is repeated and the moving image data for a plurality of volumes is output.

As described above, in the first embodiment, the data is not managed in units of volumes, but the volume data is managed as blocks of the two-dimensional ultrasonic image data of the A plane in the position in which the two-dimensional scanning is performed. For this reason, an image in the A plane displayed on the monitor 30 or an image in the A plane displayed on a monitor of the external device 2 is an image of a cross-section that is actually two-dimensionally scanned, and thus has a higher image quality than that of an MPR image of the A plane reconstructed from the volume data. In other words, conventionally, three-dimensional ultrasonic image data is reconstructed from two-dimensional ultrasonic image data of a plurality of A planes, and thereafter is processed anew by the MPR to generate the two-dimensional ultrasonic image data of the A plane. Instead, in the first embodiment, each time an A plane is two-dimensionally scanned, the two-dimensional ultrasonic image data of the A plane is generated and stored in a state ready to be output. Thus, in the first embodiment, it is possible to provide an image having a higher image quality than that conventionally available.

In particular, when a fetal heart is observed, cross-sections to be observed are a “4-chamber view”, and a “3-vessel view” and a “3-vessels and trachea view” which are approximately parallel to the “4-chamber view”. These three cross-sections are observed as A planes in the volume data. In the first embodiment, the volume data is constructed as moving image data of A planes. This allows the above-mentioned three cross-sections to be observed as higher quality images than MPR images in the A planes reconstructed from the volume data. Accordingly, in the first embodiment, it is possible to avoid the degradation in the image quality of the image displayed through the three-dimensional ultrasonic scanning.

Conventionally, data after the three-dimensional scanning is stored in management units of three-dimensional data. Thus, dedicated software for three-dimensional image is required to display two-dimensional images based on the volume data and to analyze the volume data. The three-dimensional ultrasonic diagnostic apparatus is equipped with the software for three-dimensional image. However, the workstation or the PC for image interpretation serving as the external device 2 is often not equipped with the software for three-dimensional image. For this reason, so as to make image interpretation possible on the PC operated by an image interpretation operator, the operator of the conventional ultrasonic diagnostic apparatus 100 has output, as diagnostic image data, a plurality of pieces of two-dimensional image data based on the volume data to the workstation or the PC for image interpretation, a database, a printer, a storage medium, or the like, that serves as the external device 2.

Medical image data is normally output to the external device 2 in a data format compliant with the Digital Imaging and Communications in Medicine (DICOM) standard. According to the DICOM standard, the volume data can be dealt with by using a 3D data tag as a standard tag. However, when using a 3D data tag a device on the output side needs to attach, as supplementary information, position information based on a three-dimensional coordinate system specific to the device such as an X-ray CT apparatus. However, in the three-dimensional ultrasonic diagnostic apparatus in which the ultrasonic probe 10 contacts an arbitrary position on the subject P, it is not appropriate to set the three-dimensional coordinate system specific to the device such as the X-ray CT apparatus. In addition, scanning lines of the ultrasonic diagnostic apparatus often radially extend, and thus, when 3D data is constructed, it is not necessarily efficient to arrange the data according to a orthogonal coordinate system of x, y, and z. Thus, 3D data compliant with the DICOM format is not commonly used in the ultrasonic diagnostic apparatus.

Thus, three-dimensional data or four-dimensional data collected through an ultrasonic examination has had to be attached with, for example, a private tag specific to the ultrasonic examination, and then to be output to the external device 2. In other words, the three-dimensional data generated by the three-dimensional ultrasonic scanning has been treated not as DICOM data common to all systems, but as system-specific DICOM data. In addition, as described above, even having received the three-dimensional data or the four-dimensional data, the image interpretation operator cannot perform reanalysis if a PC operated by the image interpretation operator is not equipped with the software for three-dimensional image. Thus, conventionally, the pieces of two-dimensional image data have been output to the external device 2, together with the three-dimensional reflected-wave data and the volume data. This has resulted in a large data size when the three-dimensional data in the ultrasonic examination or the four-dimensional data obtained by collecting the three-dimensional data in chronological order has been dealt with according to the DICOM standard.

In the first embodiment, however, the volume data can be treated as moving image data of two-dimensional images. In other words, in the first embodiment, the volume data can be treated in the same manner as moving image data of two-dimensional images obtained by repeatedly two-dimensionally scanning a cross-section at the same position in chronological order. In the DICOM standard, the tag for moving image data is a tag according to the standard specifications. Thus, the output controller 252 can, for example, attach the tag for moving image data to the two-dimensional image data group 2000, and output it to the external device 2. The PC of the image interpretation operator is normally equipped with a DICOM viewer. Thus, without necessity of purchasing special software, the image interpretation operator can display as a movie or display as thumbnails the two-dimensional image data group 2000 output as the volume data from the ultrasonic diagnostic apparatus 1.

Thus, the first embodiment can increase the degree of freedom regarding the use of the volume data; that is, for example, the operator of the ultrasonic diagnostic apparatus 1 can ask for a second opinion about the collected volume data from another image interpretation operator.

In the first embodiment, the three-dimensional scanning is performed not by manually swinging the ultrasonic probe for two-dimensional scanning, but by continuously performing the two-dimensional scanning at a constant speed by increments of constant distance using an oscillation mechanism of the ultrasonic probe 10. Thus, in the first embodiment, the image interpretation operator can roughly understand a positional relation, in the three-dimensional space, of the pieces of two-dimensional image data which are displayed as a movie or displayed as thumbnails.

The first embodiment treats, as one unit of performing the storage control, the range in which the position of the two-dimensional scanning on each of the predetermined cross-sections (A planes) is changed, thereby facilitating dealing with the moving image data corresponding to one volume. However, the first embodiment may be a case of treating a plurality of pieces of moving image data collected through a plurality of swings of oscillation as one unit of performing the storage control. In such a case, for example, a flag is inserted between pieces of moving image data stored in the data storage 24 to indicate that the pieces differ from each other, so that the output controller 252 can recognize the start frame and the end frame of each piece of the moving image data.

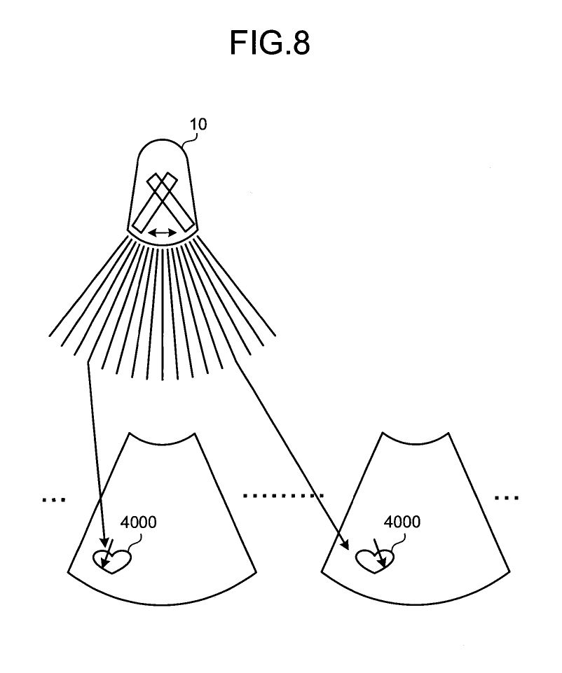

In a second embodiment, using FIGS. 6 to 8, a case will be described in which each piece of the two-dimensional image data collected as moving image data in the first embodiment is attached with information indicating a position in which the two-dimensional scanning has been performed. FIGS. 6 to 8 are diagrams for explaining the second embodiment.

The output controller 252 according to the second embodiment controls to attach the supplementary information indicating the position in which the two-dimensional scanning has been performed to each piece of the two-dimensional image data constituting a plurality of pieces of the two-dimensional image data to be output as moving image data, and to output the resultant two-dimensional image data. Specifically, the output controller 252 according to the second embodiment controls to superimpose image data based on the supplementary information onto each piece of the two-dimensional image data constituting a plurality of pieces of the two-dimensional image data, and to output the resultant two-dimensional image data.

As described in the first embodiment, the scanning conditions including the oscillation angles and the oscillation speed are set so as to start the three-dimensional scanning. The position of the A plane corresponding to each piece of the two-dimensional image data generated by the image processor 23 can be obtained from the scanning conditions. Accordingly, for example, the output controller 252 calculates, from the scanning conditions, position information of the two-dimensional image data generated by the image processor 23 in the three-dimensional scan range, as the supplementary information. Then, for example, under control of the output controller 252, the image processor 23 having a drawing function superimposes the image data based on the supplementary information onto the two-dimensional image data to generate superimposed image data. Then, under control of the output controller 252, the image processor 23 stores the superimposed image data in the data storage 24.

Then, the output controller 252 outputs a superimposed image data group for one volume as moving image data, for example, to the monitor 30 or the PC of the image interpretation operator.

The image data based on the supplementary information serves as an indicator that indicates the position of a scanned cross-section corresponding to the two-dimensional image data. In other words, an indicator with updated information on the position of the scanned cross-section is displayed in each frame of the dynamically displayed superimposed image data group as a movie.

The image data based on the supplementary information is, for example, text data for an angle (such as 26 deg or −26 deg) indicating an oscillation position, as illustrated in FIG. 6. As illustrated as an example in FIG. 6, each time the frame is updated, the text data for the angle is updated corresponding to the position of the frame.

Alternatively, the image data based on the supplementary information is, for example, image data obtained by superimposing an arrow indicating the direction of an ultrasonic beam onto an image 3000 representing the shape of the B plane in the three-dimensionally scanned range, as illustrated in FIG. 7. As illustrated as an example in FIG. 7, each time the frame is updated, the direction of the arrow superimposed on the image 3000 is updated corresponding to the position of the frame.

Still alternatively, in order to make the scan range easy to understand visually, the image data based on the supplementary information may be data using a simplified image simulating an organ to be three-dimensionally scanned. The image data based on the supplementary information is, for example, image data obtained by superimposing the arrow indicating the direction of the ultrasonic beam onto a three-dimensional body mark 4000 that is three-dimensionally drawn in the form of a heart, as illustrated in FIG. 8. As illustrated as an example in FIG. 8, each time the frame is updated, the direction of the arrow superimposed on the three-dimensional body mark 4000 is updated corresponding to the position of the frame. Note that, in order to make the scan range easier to understand visually, the above-described simplified image can be selected for each organ to be three-dimensionally scanned.

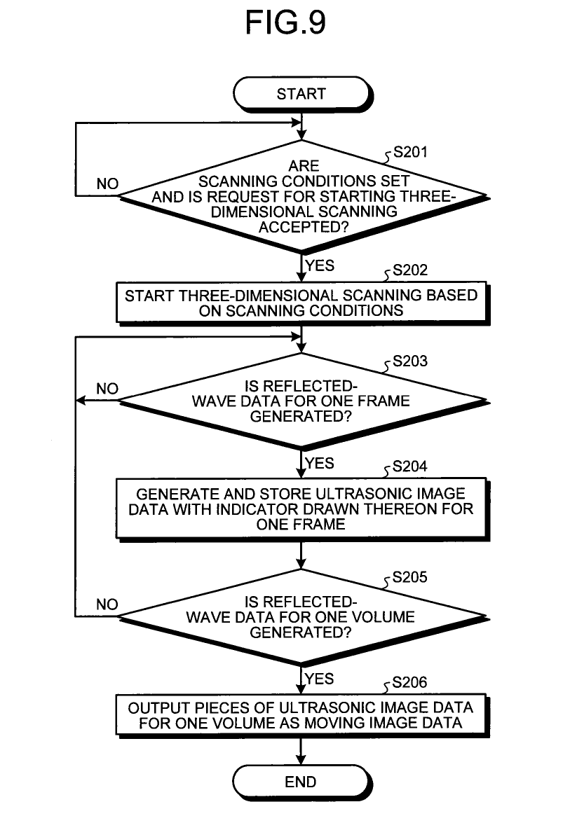

Next, processing by the ultrasonic diagnostic apparatus according to the second embodiment will be described using FIG. 9. FIG. 9 is a flowchart for explaining the processing by the ultrasonic diagnostic apparatus according to the second embodiment.

As illustrated in FIG. 9, the ultrasonic diagnostic apparatus 1 according to the second embodiment determines whether the scanning conditions are set and a request for starting the three-dimensional scanning is accepted (Step S201). If the request for starting the three-dimensional scanning is not accepted (No at Step S201), the ultrasonic diagnostic apparatus 1 waits until the request for starting the three-dimensional scanning is accepted.

If, instead, the request for starting the three-dimensional scanning is accepted (Yes at Step S201), the ultrasonic probe 10 starts, under control of the controller 250, the three-dimensional scanning based on the scanning conditions (Step S202). Then, the storage controller 251 determines whether reflected-wave data for one frame is generated (Step S203). If the reflected-wave data for one frame is not generated (No at Step S203), the storage controller 251 waits until the reflected-wave data for one frame is generated.

If, instead, the reflected-wave data for one frame is generated (Yes at Step S203), the image processor 23 generates, under control of the storage controller 251 and the output controller 252, ultrasonic image data with the indicator drawn thereon (superimposed image data) for one frame, and stores the generated data in the data storage 24 (Step S204). Then, the storage controller 251 determines whether the reflected-wave data for one volume is generated (Step S205). If the reflected-wave data for one volume is not generated (No at Step S205), the storage controller 251 returns to Step S203 and determines whether the reflected-wave data for one frame is generated.

If, instead, the reflected-wave data for one volume is generated (Yes at Step S205), the output controller 252 outputs a plurality of pieces of the ultrasonic image data for one volume (two-dimensional superimposed image data group) as moving image data (Step S206), and terminates the processing. Note that the second embodiment may be a case in which the processing from Step S203 to Step S206 is repeated, and the moving image data for a plurality of volumes is output.

As described above, in the second embodiment, the image data (indicator) indicating the position of the scanned cross-section is superimposed on each piece of the two-dimensional image data output as moving image data. In the first embodiment, the volume data is collected as moving image data composed of the pieces of two-dimensional image data of the scanned cross-sections each of whose positions is continuously changed. Also, in the first embodiment, the three-dimensional scanning is performed by performing the two-dimensional scanning at a constant speed by increments of constant distance using the oscillation mechanism of the ultrasonic probe 10. Accordingly, in the first embodiment, the image interpretation operator can roughly understand the positional relation, in the three-dimensional space, of the pieces of two-dimensional image data. In the first embodiment, however, the image interpretation operator cannot accurately understand the positional relation, in the three-dimensional space, of the pieces of two-dimensional image data. Because the ultrasonic diagnostic apparatus 1 outputs the volume data as moving image data, the image interpretation operator, who uses the PC as the external device 2, cannot recognize that the received moving image data is the volume data.

In contrast, in the second embodiment, the moving image data is output with the indicator embedded therein. Accordingly, the image interpretation operator can recognize that the received moving image data is data corresponding to the volume data, and further can easily and accurately understand the positional relation, in the three-dimensional space, of the movie displayed pieces of two-dimensional image data. Note that, in the case of superimposing the text data for an angle exemplified in FIG. 6, it is desirable to use also an indicator for indicating the position of the frame during movie display on a normal viewer, so as to increase an amount of information provided to the observer.

In the second embodiment, if a device on the receiving side is provided with a function to read supplementary information and to draw image data based on the read supplementary information, the output controller 252 may control to attach the supplementary information indicating the position in which the two-dimensional scanning has been performed to each piece of the two-dimensional image data, and to output the resultant two-dimensional image data. The image position information in the supplementary information enables reconstruction of three-dimensional image from the two-dimensional data or the two-dimensional image data, in post-processing.

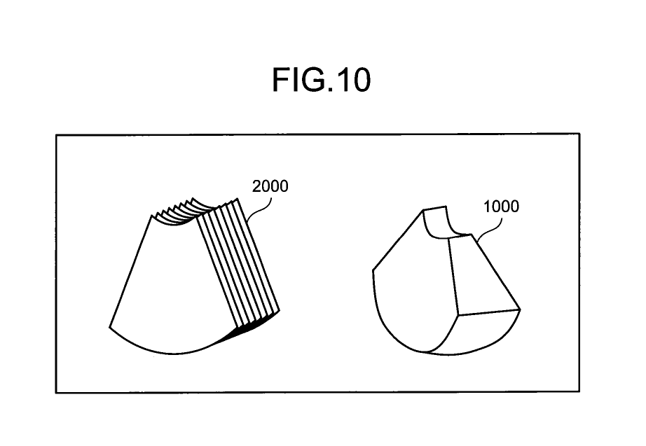

In a third embodiment, using FIG. 10, a case will be described in which data for one volume is collected as a plurality of pieces of two-dimensional data while one piece of three-dimensional data is also collected. FIG. 10 is a diagram for explaining the third embodiment.

The storage controller 251 according to the third embodiment further controls the three-dimensional data generated by the three-dimensional scanning so as to be stored in the data storage 24. Then, the output controller 252 according to the third embodiment further controls the three-dimensional image data based on the three-dimensional data stored in the data storage 24 so as to be output to the predetermined output unit (the monitor 30 or the external device 2).

In other words, the third embodiment additionally treats the three-dimensional reflected-wave data, the three-dimensional signal-processed data, and the volume data (three-dimensional ultrasonic image data) as data to be controlled to be stored. For example, in the third embodiment, when data is collected by the three-dimensional scanning, the image processor 23 generates and stores, under control of the storage controller 251, the moving image data composed of the pieces of two-dimensional image data described in the first and second embodiments, and in addition, the normal three-dimensional data (such as the three-dimensional ultrasonic image data). Consequently, the data storage 24 stores therein, for example, as illustrated in FIG. 10, the volume data 1000, in addition to the two-dimensional image data group 2000.

The output controller 252 outputs, for example, the two-dimensional image data group 2000 stored as DICOM image data to the external device 2. The output controller 252 also outputs, for example, the volume data 1000 as DICOM 3D data attached with a private tag to the external device 2 equipped with the software for three-dimensional image. Note the third embodiment may be a case in which the data storage 24 stores therein the three-dimensional reflected-wave data and the three-dimensional signal-processed data, and when an output request for three-dimensional data is issued, the apparatus body 200 generates volume data and outputs the generated volume data.

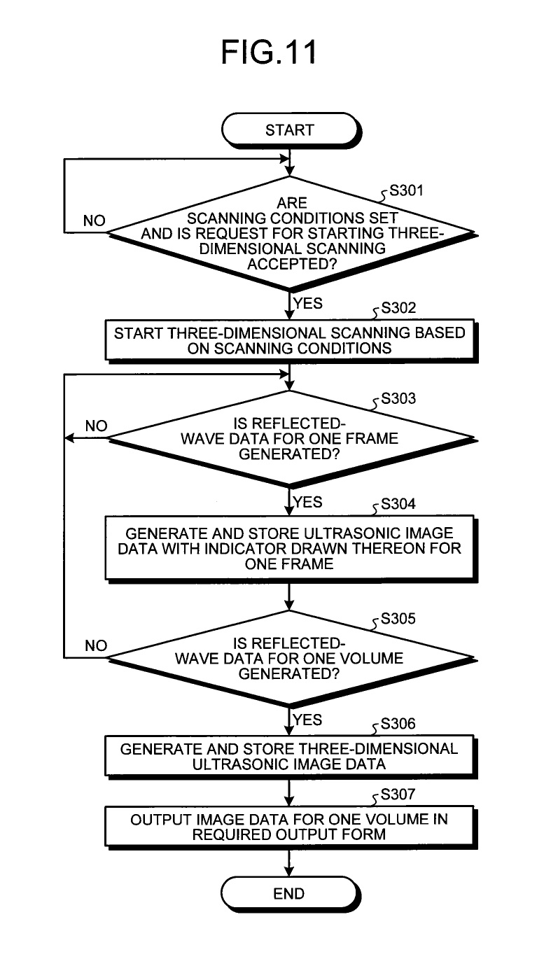

Next, processing by an ultrasonic diagnostic apparatus according to the third embodiment will be described using FIG. 11. FIG. 11 is a flowchart for explaining the processing by the ultrasonic diagnostic apparatus according to the third embodiment. Note that, in the flowchart described below, a case will be described in which the three-dimensional ultrasonic image data is to be stored as the three-dimensional data. Also, in the flowchart described below, a case will be described in which the indicator described in the second embodiment is superimposed on the two-dimensional ultrasonic image data. However, the third embodiment may be a case in which no indicator is superimposed as described in the first embodiment.

As illustrated in FIG. 11, this ultrasonic diagnostic apparatus 1 according to the third embodiment determines whether the scanning conditions are set and a request for starting the three-dimensional scanning is accepted (Step S301). If the request for starting the three-dimensional scanning is not accepted (No at Step S301), the ultrasonic diagnostic apparatus 1 waits until the request for starting the three-dimensional scanning is accepted.

If, instead, the request for starting the three-dimensional scanning is accepted (Yes at Step S301), the ultrasonic probe 10 starts, under control of the controller 250, the three-dimensional scanning based on the scanning conditions (Step S302). Then, the storage controller 251 determines whether reflected-wave data for one frame is generated (Step S303). If the reflected-wave data for one frame is not generated (No at Step S303), the storage controller 251 waits until the reflected-wave data for one frame is generated.

If, instead, the reflected-wave data for one frame is generated (Yes at Step S303), the image processor 23 generates, under control of the storage controller 251 and the output controller 252, ultrasonic image data with the indicator drawn thereon (superimposed image data) for one frame, and stores the generated data in the data storage 24 (Step S304). Then, the storage controller 251 determines whether the reflected-wave data for one volume is generated (Step S305). If the reflected-wave data for one volume is not generated (No at Step S305), the storage controller 251 returns to Step S303 and determines whether the reflected-wave data for one frame is generated.

If, instead, the reflected-wave data for one volume is generated (Yes at Step S305), the image processor 23 generates, under control of the storage controller 251, the three-dimensional ultrasonic image data (volume data), and stores the generated data in the data storage 24 (Step S306).

Then, the output controller 252 outputs the image data for one volume (at least one of the moving image data and the volume data) in a required output form (Step S307), and terminates the processing. Note that the third embodiment may be a case in which the processing from Step S303 to Step S307 is repeated, and the image data for a plurality of volumes is output.

As described above, in the third embodiment, the moving image data and the volume data are generated as data corresponding to one volume. That is to say, the third embodiment allows the operator of the external device 2 equipped with the software for three-dimensional image to display the image data based on the volume data and to analyze the volume data. Thus, the third embodiment can further increase the degree of freedom regarding the use of the volume data.

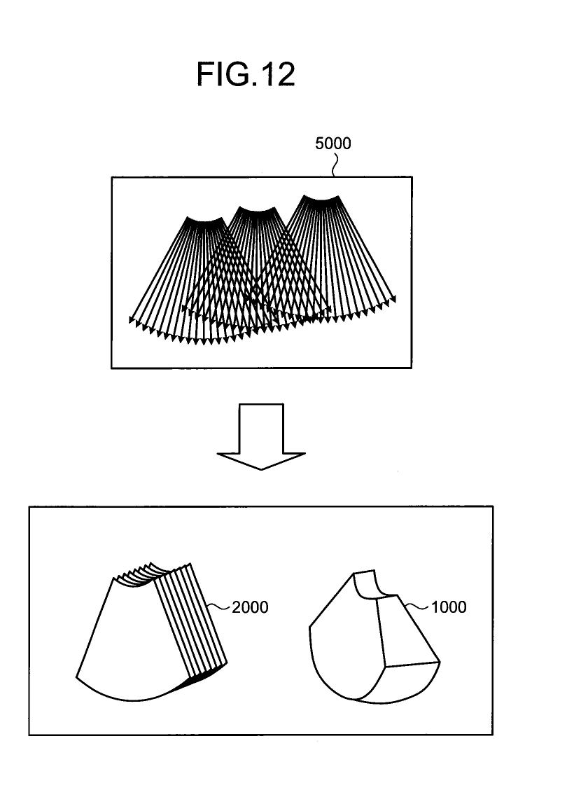

In a fourth embodiment, using FIG. 12, a case will be described in which, instead of the two-dimensional image data, the two-dimensional reflected-wave data is treated as two-dimensional data to be controlled to be stored. FIG. 12 is a diagram for explaining the forth embodiment.

The storage controller 251 according to the fourth embodiment controls a plurality of pieces of two-dimensional reflected-wave data so as to be stored in the data storage 24 as the pieces of two-dimensional data.

The output controller 252 according to the fourth embodiment controls at least one of the pieces of two-dimensional image data based on the pieces of two-dimensional reflected-wave data and the three-dimensional image data based on the pieces of two-dimensional reflected-wave data so as to be output to the predetermined output unit (the monitor 30 or the external device 2).

In other words, the storage controller 251 controls to store, as one unit in the data storage 24, the two-dimensional reflected-wave data group generated by the transmitter/receiver 21, each time the position of the two-dimensional scanning to be changed is changed. Consequently, the data storage 24 stores therein, for example, as illustrated in FIG. 12, two-dimensional reflected-wave data group 5000 which is the pieces of two-dimensional reflected-wave data each corresponding to one of the A planes constituting the three-dimensionally scanned region. The two-dimensional reflected-wave data group 5000 is processed through the processing by the signal processor 22 and the image processor 23, so as to be generated as the two-dimensional image data group 2000 and the volume data 1000, as illustrated in FIG. 12.

Note that, because the position information of the scanning cross-section of each of the pieces of two-dimensional reflected-wave data can be calculated from the scanning conditions, there may be a case in which the two-dimensional image data group 2000 is generated as the superimposed image data group described in the second embodiment.

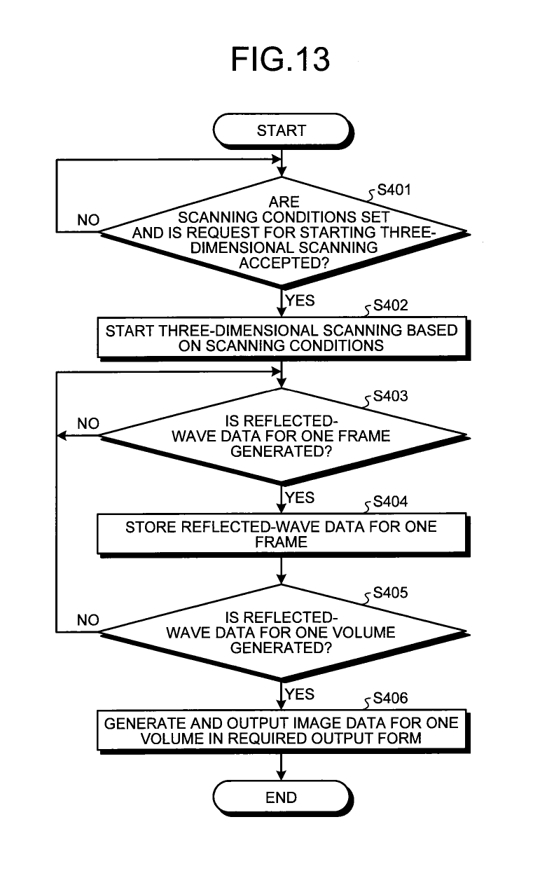

Next, processing by an ultrasonic diagnostic apparatus according to the fourth embodiment will be described using FIG. 13. FIG. 13 is a flowchart for explaining the processing by the ultrasonic diagnostic apparatus according to the fourth embodiment. Note that, in the flowchart described below, a case will be described in which no indicator is superimposed as described in the first embodiment. However, the fourth embodiment may be a case in which the indicator described in the second embodiment is superimposed on the two-dimensional ultrasonic image data.

As illustrated in FIG. 13, this ultrasonic diagnostic apparatus 1 according to the fourth embodiment determines whether the scanning conditions are set and a request for starting the three-dimensional scanning is accepted (Step S401). If the request for starting the three-dimensional scanning is not accepted (No at Step S401), the ultrasonic diagnostic apparatus 1 waits until the request for starting the three-dimensional scanning is accepted.

If, instead, the request for starting the three-dimensional scanning is accepted (Yes at Step S401), the ultrasonic probe 10 starts, under control of the controller 250, the three-dimensional scanning based on the scanning conditions (Step S402). Then, the storage controller 251 determines whether reflected-wave data for one frame is generated (Step S403). If the reflected-wave data for one frame is not generated (No at Step S403), the storage controller 251 waits until the reflected-wave data for one frame is generated.

If, instead, the reflected-wave data for one frame is generated (Yes at Step S403), the transmitter/receiver 21 stores, under control of the storage controller 251, the reflected-wave data (two-dimensional reflected-wave data) for one frame in the data storage 24 (Step S404). Then, the storage controller 251 determines whether the reflected-wave data for one volume is generated (Step S405). If the reflected-wave data for one volume is not generated (No at Step S405), the storage controller 251 returns to Step S403, and determines whether the reflected-wave data for one frame is generated.

If, instead, the reflected-wave data for one volume is generated (Yes at Step S405), the signal processor 22 and the image processor 23 generate and output, under control of the output controller 252, the image data for one volume (at least one of the moving image data and the volume data) in a required output form (Step S406), and terminates the processing. Note that the fourth embodiment may be a case in which the processing from Step S403 to Step S406 is repeated, and the image data for a plurality of volumes is output.

As described above, in the fourth embodiment, the control is performed so as to store the pieces of two-dimensional reflected-wave data from which both the moving image data and the volume data can be generated. When, for example, the processing of the third embodiment is performed, the moving image data and the volume data are stored as data corresponding to one volume in the data storage 24, thereby expanding the data size. However, in the fourth embodiment, the pieces of two-dimensional reflected-wave data are stored, and thus, the size of the data stored in the data storage 24 can be reduced.

In addition, in the fourth embodiment, in response to the required output form, at least one of the moving image data and the volume data can be quickly generated and output as data corresponding to one volume.

Moreover, the fourth embodiment is useful particularly when the three-dimensional scanning is performed in a scan sequence in which both the color Doppler mode and the B mode are used. Specifically, if a display image of a B-mode image with a color Doppler image superimposed thereon is stored as it is, it is difficult to switch between display and non-display of the color Doppler image in post-processing. However, in the fourth embodiment in which the pieces of two-dimensional reflected-wave data are stored, when, for example, moving image data is generated, it is possible to generate and output moving image data that is arbitrarily switched between “with the color Doppler image” and “without the color Doppler image” in response to the output form requested by the operator. In other words, in the fourth embodiment in which the pieces of two-dimensional reflected-wave data are stored, it is possible to change or adjust, in units of cross-section, the moving image data generated and output in the post-processing.

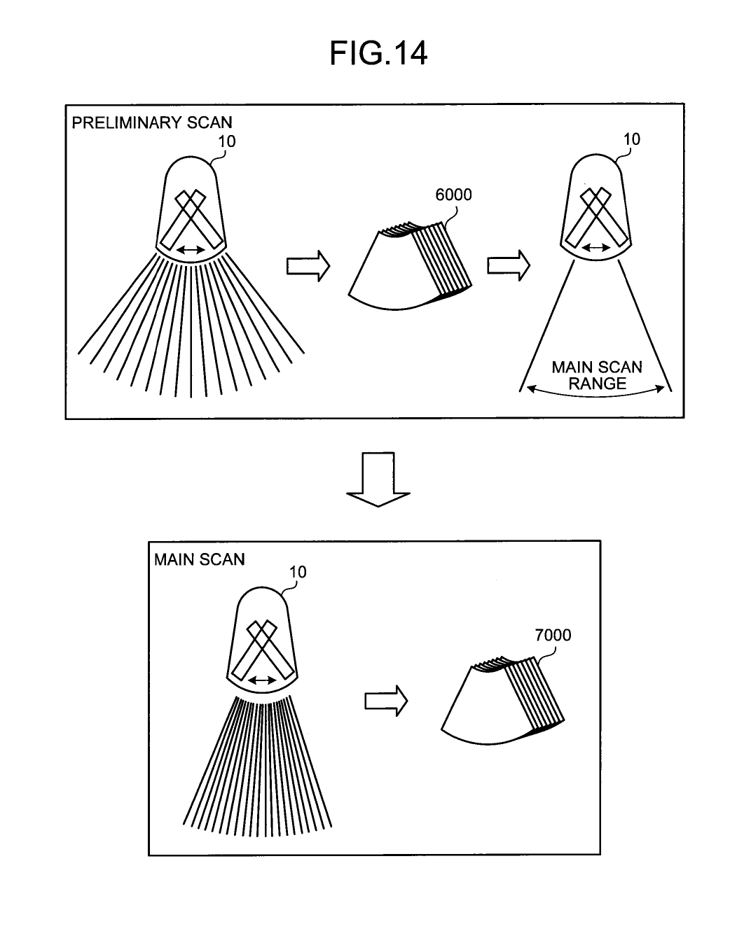

In a fifth embodiment, using FIG. 14, a case will be described in which, when the three-dimensional scanning is performed through the mechanical oscillation, a preliminary scan is performed, and thereafter, scanning conditions for a main scan are determined.

In the fifth embodiment, the input device 40 accepts preliminary scanning conditions for preliminary scan. The preliminary scanning conditions are set, for example, as follows: the range of the preliminary scan is set to the maximum range of the mechanical oscillation of the ultrasonic probe 10; the oscillation speed is set higher; and the interval between the collection cross-sections is set larger. For example, the interval between the collection cross-sections is set to two degrees, and the oscillation time for one swing is set to five seconds. Note that the preliminary scanning conditions may be initially set in advance.

After the preliminary scanning conditions are set and a request for starting the three-dimensional scanning is accepted, the ultrasonic probe 10 performs the preliminary scan as illustrated in FIG. 14. After the preliminary scan is finished, moving image data 6000 of the preliminary scan illustrated in FIG. 14 is controlled by the output controller 252 to be displayed on the monitor 30. The moving image data 6000 is the pieces of two-dimensional image data described in the first embodiment or the pieces of superimposed image data described in the second embodiment.

Then, the input device 40 accepts changes of the scanning conditions from the operator who has referred, as moving image data, to the pieces of two-dimensional image data or the pieces of superimposed image data based on the pieces of two-dimensional image data. The operator, who has referred to the pieces of superimposed image data as the moving image, refers to the indicators to identify the oscillation angles including therebetween a region of interest, and enters the identified oscillation angles via the input device 40.

Alternatively, the operator who has referred to the pieces of superimposed image data or the pieces of two-dimensional image data as thumbnails specifies, via the input device 40, two pieces of image data that serve as boundaries of a range including a region of interest. Then, for example, the output controller 252 calculates the positions of scanned cross-sections of the specified two pieces of two-dimensional image data from the preliminary scanning conditions. With this, as illustrated in FIG. 14, the controller 250 can set a main scan range (oscillation angles of the main scan) defined by a start angle and a finish angle. Note that the interval between the collection cross-sections and the oscillation speed for the main scan is set by the operator. As illustrated in FIG. 14, the main scan range is normally set narrower than the preliminary scan range. Accordingly, the operator sets the interval between the collection cross-sections and the oscillation speed so as to increase the scanning line density in the direction of oscillation within the limit of not reducing the volume rate.

Then, after the main scanning conditions are set and a request for starting the three-dimensional scanning is accepted, the ultrasonic probe 10 performs the main scan as illustrated in FIG. 14. After the main scan is finished, moving image data 7000 of the main scan illustrated in FIG. 14 is stored in the data storage 24. Then, the moving image data 7000 of the main scan is, for example, displayed on the monitor 30 as a movie. Note that, in the fifth embodiment, the modes of the storage control and the output control described in any one of the first to fourth embodiments can be selected as modes of storage control and output control in the main scan.

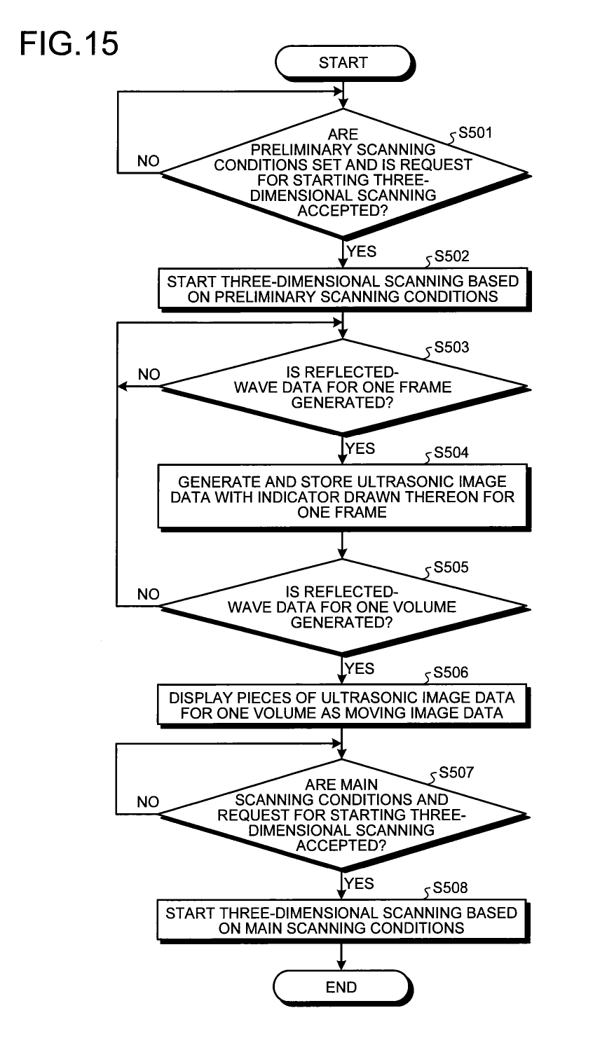

Next, processing by an ultrasonic diagnostic apparatus according to the fifth embodiment will be described using FIG. 15. FIG. 15 is a flowchart for explaining the processing by the ultrasonic diagnostic apparatus according to the fifth embodiment. Note that, in the flowchart described below, a case will be described in which the indicator described in the second embodiment is superimposed on the two-dimensional ultrasonic image data. However, the fifth embodiment may be a case in which no indicator is superimposed as described in the first embodiment.

As illustrated in FIG. 15, this ultrasonic diagnostic apparatus 1 according to the fifth embodiment determines whether the preliminary scanning conditions are set and a request for starting the three-dimensional scanning is accepted (Step S501). If the request for starting the three-dimensional scanning is not accepted (No at Step S501), the ultrasonic diagnostic apparatus 1 waits until the request for starting the three-dimensional scanning is accepted.

If, instead, the request for starting the three-dimensional scanning is accepted (Yes at Step S501), the ultrasonic probe 10 starts, under control of the controller 250, the three-dimensional scanning based on the preliminary scanning conditions (Step S502). Then, the storage controller 251 determines whether reflected-wave data for one frame is generated (Step S503). If the reflected-wave data for one frame is not generated (No at Step S503), the storage controller 251 waits until the reflected-wave data for one frame is generated.

If, instead, the reflected-wave data for one frame is generated (Yes at Step S503), the image processor 23 generates, under control of the storage controller 251, ultrasonic image data with the indicator drawn thereon (superimposed image data) for one frame, and stores the generated data in the data storage 24 (Step S504). Then, the storage controller 251 determines whether the reflected-wave data for one volume is generated (Step S505). If the reflected-wave data for one volume is not generated (No at Step S505), the storage controller 251 returns to Step S503 and determines whether the reflected-wave data for one frame is generated.

If, instead, the reflected-wave data for one volume is generated (Yes at Step S505), the monitor 30, under control of the output controller 252, displays a plurality of pieces of the ultrasonic image data for one volume (two-dimensional superimposed image data group) as moving image data (Step S506).

Then, the controller 250 determines whether the main scanning conditions and a request for starting the three-dimensional scanning are accepted from the input device 40 (Step S507). If the main scanning conditions and the request for starting the three-dimensional scanning are not accepted (No at Step S507), the ultrasonic diagnostic apparatus 1 waits until the main scanning conditions and the request for starting the three-dimensional scanning are accepted.

If, instead, the main scanning conditions and the request for starting the three-dimensional scanning are accepted (Yes at Step S507), the ultrasonic probe 10 starts, under control of the controller 250, the three-dimensional scanning based on the main scanning conditions (Step S508), and terminates the processing. Note that, after the processing of Step S508, the storage control and the output control described in any one of the first to fourth embodiments are performed.

As described above, in the fifth embodiment, the moving image data corresponding to one volume is displayed so as to facilitate the setting of the main scanning conditions for focusing the scanning on a region of interest.

Note that the first to fifth embodiments described above are even applicable to a case in which the ultrasonic probe 10 performs the three-dimensional scanning based on the transmission/reception control using a two-dimensionally arranged vibrator group. In other words, the first to fifth embodiments described above are even applicable to a case where the ultrasonic probe 10 uses a 2D probe with the piezoelectric vibrators arranged in a matrix state that can three-dimensionally scan the subject P with ultrasonic waves. The 2D probe can also two-dimensionally scan the probe P by transmitting the ultrasonic waves in a focused manner, and thus can perform the three-dimensional scanning by continuously moving the position of the A plane in the direction of oscillation as is performed by the mechanical 4D probe.

There may be a case in which the image processing method described in each of the first to fifth embodiments is performed by an image processing apparatus installed independently of the ultrasonic diagnostic apparatus 1. Such an image processing apparatus can perform the image processing method described in each of the first to fifth embodiments, for example, by receiving the reflected-wave data generated by the transmitter/receiver 21.

The constituent elements of the devices illustrated in the drawings are functionally conceptual, and need not be physically configured as illustrated. In other words, the specific mode of separation and integration of the devices is not limited to those illustrated in the drawings, but all or part of the devices can be configured to be separated or integrated either functionally or physically in any units according to various types of loads or use conditions. Further, all or any part of the processing functions performed in the devices can be implemented with a CPU and a program analyzed and executed by the CPU, or can be implemented as hardware with a wired logic.

The image processing method described in each of the first to fifth embodiments can be implemented by executing an image processing program provided in advance on a computer such as a personal computer or a workstation. The image processing program can be distributed via a network such as the Internet. This image processing program can also be executed by being recorded on a computer-readable non-transitory recording medium, such as a flexible disk (FD), a CD-ROM, an MO, a DVD, a USB memory, or a flash memory such as an SD card memory, and by being read by a computer from the non-transitory recording medium.

As described above, according to the first to fifth embodiments, it is possible to avoid degradation in image quality of an image displayed through the three-dimensional ultrasonic scanning.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
an ultrasonic probe configured to perform three-dimensional ultrasonic scanning under transmission/reception control;
a storage controller configured to control data generated by the three-dimensional scanning performed by the ultrasonic probe so as to be stored in a predetermined memory as a plurality of pieces of two-dimensional data generated by two-dimensionally scanning a plurality of predetermined cross-sections each of whose positions is continuously changed along a predetermined direction in a region of the three-dimensional scanning; and
an output controller configured to control a plurality of pieces of two-dimensional image data based on the pieces of two-dimensional data stored in the predetermined memory so as to be output as moving image data to a predetermined output unit.

2. The ultrasonic diagnostic apparatus according to claim 1, wherein the output controller is configured to control to attach supplementary information indicating the position in which the two-dimensional scanning has been performed to each piece of the two-dimensional image data constituting the pieces of two-dimensional image data, and to output the resultant two-dimensional image data.

3. The ultrasonic diagnostic apparatus according to claim 2, wherein the output controller is configured to control to superimpose image data based on the supplementary information onto each piece of the two-dimensional image data constituting the pieces of two-dimensional image data, and to output the resultant two-dimensional image data.

4. The ultrasonic diagnostic apparatus according to claim 3, wherein the image data based on the supplementary information is data using a simplified image simulating an organ to be three-dimensionally scanned.

5. The ultrasonic diagnostic apparatus according to claim 4, wherein the simplified image is selectable for each organ to be three-dimensionally scanned.

6. The ultrasonic diagnostic apparatus according to claim 1, wherein
the storage controller is configured to control the pieces of two-dimensional image data so as to be stored in the predetermined memory as the pieces of two-dimensional data; and
the output controller is configured to control the pieces of two-dimensional image data stored in the predetermined memory so as to be output as moving image data to the predetermined output unit.

7. The ultrasonic diagnostic apparatus according to claim 1, wherein
the storage controller is configured to further control three-dimensional data generated by the three-dimensional scanning so as to be stored in the predetermined memory; and
the output controller is configured to further control three-dimensional image data based on the three-dimensional data stored in the predetermined memory so as to be output to the predetermined output unit.

8. The ultrasonic diagnostic apparatus according to claim 1, wherein
the storage controller is configured to control a plurality of pieces of two-dimensional reflected-wave data so as to be stored in the predetermined memory as the pieces of two-dimensional data; and
the output controller is configured to control at least one of a plurality of pieces of two-dimensional image data based on the pieces of two-dimensional reflected-wave data and three-dimensional image data based on the pieces of two-dimensional reflected-wave data so as to be output to the predetermined output unit.

9. The ultrasonic diagnostic apparatus according to claim 1, wherein the position of each of the predetermined cross-sections is changed by increments of constant amount.

10. The ultrasonic diagnostic apparatus according to claim 1, further comprising an input unit configured to accept changes in scanning conditions from an operator who has referred to the pieces of two-dimensional image data as moving image data.

11. The ultrasonic diagnostic apparatus according to claim 1, wherein the storage controller is configured to treat, as one unit of performing the storage control, a range in which the position of the two-dimensional scanning on each of the predetermined cross-sections is changed.

12. The ultrasonic diagnostic apparatus according to claim 1, wherein the ultrasonic probe is configured to perform the three-dimensional scanning by oscillating a vibrator group arranged in a line.

13. The ultrasonic diagnostic apparatus according to claim 1, wherein the ultrasonic probe is configured to perform the three-dimensional scanning using a two-dimensionally arranged vibrator group.

14. An image processing apparatus comprising:
a storage controller configured to control data generated by three-dimensional scanning performed by an ultrasonic probe under transmission/reception control so as to be stored in a predetermined memory as a plurality of pieces of two-dimensional data generated by two-dimensionally scanning a plurality of predetermined cross-sections each of whose positions is continuously changed along a predetermined direction in a region of the three-dimensional scanning; and
an output controller configured to control a plurality of pieces of two-dimensional image data based on the pieces of two-dimensional data stored in the predetermined memory so as to be output as moving image data to a predetermined output unit.

15. An image processing method comprising:
controlling, by a storage controller, data generated by three-dimensional scanning performed by an ultrasonic probe under transmission/reception control so as to be stored in a predetermined memory as a plurality of pieces of two-dimensional data generated by two-dimensionally scanning a plurality of predetermined cross-sections each of whose positions is continuously changed along a predetermined direction in a region of the three-dimensional scanning; and
controlling, by an output controller, a plurality of pieces of two-dimensional image data based on the pieces of two-dimensional data stored in the predetermined memory so as to be output as moving image data to a predetermined output unit.
